(19) United States
(12) Patent Application Publication
(10) Pub. No.: US 2016/0256540 A1
(43) Pub. Date: Sep. 8, 2016

(54) Methods And Compositions For Treatment Of S. Equi Infection

(71) Applicant: ZOETIS SERVICES LLC, Florham Park, NJ (US)

(72) Inventors: Ellen Ons, Heverlee (BE); Rudiger Raue, Winksele (BE)

(21) Appl. No.: 15/028,814

(22) PCT Filed: Oct. 15, 2014
(86) PCT No.: PCT/US2014/060597

§ 371 Date: Apr. 12, 2016

Related U.S. Application Data
(60) Provisional application No. 61/892,080, filed on Oct. 17, 2013.

(51) Int. Cl. A61K 39/275

(52) U.S. Cl. CPC A61K 39/275; C12N 2710/24234; A61K 2039/5252

(57) ABSTRACT

The instant invention provides methods for treatment or prevention of S equi and/or EHV in horses, the methods comprising administered to the horses in need thereof a com position comprising parapoxvirus ovis.

### FIELD OF INVENTION

The instant invention relates to methods for treating Streptococcus equi subsp. equi (Strep equi) in horses.

### BACKGROUND

Strep equi, which is virtually confined to horses, is the causative agent of strangles, a world-wide distributed and highly contagious serious disease of the upper respiratory tract of the Equidae. Strangles is an acute upper respiratory tract disease of horses. This highly contagious disease is characterized by fever, nasal discharge and abscess formation in the retropharyngeal and mandibular lymph nodes. The swelling of the lymph nodes is frequently so severe that the animal airways become obstructed. Morbidity is generally high, and can be as high as 100%, in susceptible populations.

Although it is in principle possible to treat and cure these streptococcal infections with antibiotics, such as penicillin, tetracycline or gentamicin, antibiotics may not always be used since studies have shown that antimicrobials cannot easily penetrate the abscess capsule present in the infection. Therefore, treatment often revolves around supportive care, good stable management, and hygiene. An effective prophylactic and/or therapeutic agent that could prevent or reduce outbursts of such infections and obviate, and/or reduce the risk of the development of resistant strains associated with antibiotic treatment, would be appreciated.

Horses infected with strangles (in the field or experimentally), which recover from the disease become highly resistant to re-infection. In view of this fact, attempts have been made to develop an effective and safe vaccine against strangles. For example, vaccines prepared from bacterins of Strep equi, or fractional extracts thereof, such as M protein-rich extracts, have been developed. However, the existing vaccine compositions are not completely satisfactory. Some are relatively ineffective at providing protection against Strep equi in the field and others have side effects. One of the problems with this line of research was that scientists tried to induce protection against Strep equi by stimulating bactericidal antibodies in the blood serum of the horse.

Two groups of researches have reported that vaccination may require stimulation of the nasopharyngeal immune response using a live Strep equi. Timoney et al. (U.S. Pat. No. 5,183,659) have prepared a composition adapted for nasal and oral administration which contained a non-encapsulated avirulent strain of Strep equi suspended in Todd Hewit broth.

Another group of researchers (EP 786,518) prepared a composition for nasal administration containing an encapsulated Strep equi strain TW928 having an unidentified 1 kb deletion in its genome. This composition, however, was not tested for its effectiveness in horses. Therefore, there is still a need in the art for effective and safe vaccines against Strep equi, particularly those that can be safely administered to young horses.

### SUMMARY OF INVENTION

The instant invention addresses these and other drawbacks of the prior art by providing, in the first aspect, a method of protecting a horse in need thereof against a Strep equi infection comprising administering to said horse an immunologically effective amount of Parapoxvirus ovis.

In the second aspect, the invention provides a method of protecting a horse against concurrent Strep equi and EHV infections, comprising administering to said horse an immunologically effective amount of Parapoxvirus ovis.

In different embodiments, the Parapoxvirus ovis may be modified live or an inactivated Parapoxvirus ovis.

In different embodiments applicable to either aspect described above, the Parapoxvirus ovis comprises Parapoxvirus ovis strain D1701.

In certain embodiments, the Parapoxvirus ovis is administered in an aqueous composition, which does not contain an adjuvant.

In certain embodiments, the compositions of the instant invention may be administered, three times, wherein the first administration precedes the second administration by about two days, and the second administration precedes the third administration by about 2 to about 10 days. In one embodiment, the second administration precedes the third administration by about 7 days.

### DETAILED DESCRIPTION OF SELECTED EMBODIMENTS

For the purposes of a better understanding of the instant invention, the following non-limiting definitions are provided:

The terms “about” or “approximately,” when used in connection with a measurable numerical variable, refers to the indicated value of the variable and to all values of the variable that are within the experimental error of the indicated value (e.g., within the 95% confidence interval for the mean) or within 10 percent of the indicated value, whichever is greater.

The term “antibody” refers to an immunoglobulin molecule that can bind to a specific antigen as the result of an immune response to that antigen. Immunoglobulins are serum proteins composed of “light” and “heavy” polypeptide chains having “constant” and “variable” regions and are divided into classes (e.g., IgA, IgD, IgE, IgG, and IgM) based on the composition of the constant regions.

The term “buffer” means a chemical system that prevents change in the concentration of another chemical substance, e.g., proton donor and acceptor systems serve as buffers preventing marked changes in hydrogen ion concentration (pH). A further example of a buffer is a solution containing a mixture of a weak acid and its salt (conjugate base) or a weak base and its salt (conjugate acid).

The term “effectively immunized” refers to susceptibility or a lack thereof to a specific antigen. Thus, a horse is not “effectively immunized” against Strep equi when the horse is susceptible to Strep equi infection. Such situation may occur, for example, when the horse has not been immunized against Strep equi at all, or when the immunization regimen is incomplete, or when the protective effect of the vaccination has expired (i.e., the horse is past Duration of Immunity for the vaccination).

The term “immunologically protective amount” or “immunologically effective amount” or “effective amount to produce an immune response” of an antigen is an amount effective to induce an immunogenic response in the recipient. The immunogenic response may be sufficient for diagnostic purposes or other testing, or may be adequate to prevent signs or symptoms of disease, including adverse health effects or complications thereof, caused by infection with a disease agent. Either humoral immunity or cell-mediated immunity or both may be induced.

The immunogenic response of an animal to an immunogenic composition may be evaluated, e.g., indirectly through measurement of antibody titers, lymphocyte proliferation assays, or directly through monitoring signs and symptoms after challenge with wild type strain, whereas the protective immunity conferred by a vaccine can be evaluated by measuring, e.g., reduction in clinical signs such as mortality, morbidity, temperature number, overall physical condition, and overall health and performance of the subject. The immune response may comprise, without limitation, induction of cellular and/or humoral immunity.

The term “pharmaceutically acceptable” refers to substances, which are within the scope of sound medical judgment, suitable for use in contact with the tissues of subjects without undue toxicity, irritation, allergic response, and the like, commensurate with a reasonable benefit-to-risk ratio, and effective for their intended use.

The term “treating” refers to preventing a disorder, condition, or disease to which such term applies, or to preventing or reducing one or more symptoms of such disorder, condition, or disease.

The inventors have surprisingly discovered that it is possible to successfully treat Strep equi infection without antibiotics and without using a specific vaccine against Strep equi. As described in greater details below, administration of parapoxvirus was effective against Strep equi infection. The parapoxvirus ovis useful in the instant invention may be a modified live virus or an inactivated virus. Conveniently, methods of preparing modified live or inactivated viruses are well known in the art and straightforward.

Multiple strains of parapoxvirus are suitable for the instant invention. A person of ordinary skill in the art would appreciate that many parapoxvirus strains have been disclosed in public databases, including, without limitations, Genbank and/or deposited into well-known deposit collections, such as, for example, ATCC. In some exemplary non-limiting embodiments, the suitable strains include D1701, NZ-1, NZ-2, BO15, Orf-11, IA-82, SA00 and combinations thereof.

In certain embodiments, the Parapoxvirus ovis is Parapoxvirus ovis strain D1701. Inactivated Parapoxvirus ovis strain D1701 has been long known and is currently on the market under the trade name ZYLEXIS®. ZYLEXIS® is an immune modulator that aids in the reduction of upper respiratory disease associated with equine herpesvirus EHV-1 and/or EHV-4. Common stressors, including trailering, competition, breeding and environmental changes can trigger EHV. ZYLEXIS® may be administered before stressful situations and during disease episodes to stimulate immune response.

The exact mechanism of action of Parapoxvirus ovis is not fully understood but may involve the stimulation and increase of the non-specific immune mechanisms. In a mouse model, it has been demonstrated that Parapoxvirus ovis induces an autoregulatory cytokine response that involves the up regulation of T helper (Th) 1 type cytokines (IL-12, IL-18, IFNγ) and their subsequent down regulation which is accompanied by induction of IL-4. Furthermore, Parapoxvirus ovis induces phagocytic activity and oxidative burst in various animal species including horses as demonstrated by ex vivo experiments. In horses, it has been shown that administration of the product stimulates the proliferation of lymphocytes and increases the production of IFNy in vivo. It was also shown that administration of the product to horses increases the production of other cytokines such as TNFα, IFN β, IL15 and IL18 in vivo.

ZYLEXIS® is provided as a two-component medicine containing a pre-determined amount of freeze-dried Parapoxvirus ovis D1701 which generates a minimum of 1 relative potency (RP) per dose, and 2 ml of sterile water for injection as diluent. The components are to be mixed before the use. Thus, in different embodiments of the invention one dose of inactivated parapoxvirus ovis contains an amount which generates between 1 RP per dose and about 11.6 RP per dose.

In different embodiments of the instant invention, different diluents may be used, including pharmaceutically acceptable buffers, including, without limitation, phosphate-based buffers, saline, etc. Alternatively, the freeze-dried or lyophilized Parapoxvirus ovis may be reconstituted in water for injection. Preferably, the compositions of the instant invention are formulated for intramuscular injections, but may also be administered subcutaneously, intra-nasally or by nebulisation.

It is recommended that ZYLEXIS® should be administered to horses in three administrations, namely, on day zero, day 2 and day 9. Thus, in certain embodiments, the interval between the first and the second administration parapoxvirus ovis is generally about two days (e.g., between about 44 and about 56 hours), and the interval between the second and the third administration may vary from about two days to about ten days (i.e., about 3 days, about 4 days, about 5 days about 6 days, about 7 days, about 8, about 9 days). In a particular embodiment, the interval between the second and the third administration is about seven days. In a particular embodiment, follow on administrations are every about 2 to 7 days (i.e., about 2 days, about 3 days, about 4 days, about 5 days about 6 days, about 7 days).

An adjuvant may be added to the Parapoxvirus ovis composition of the instant invention. Suitable adjuvants are described, for example, in U.S. publications 20050191308, 20090324641, 20050220814, 20130084306, 20100047279. However, in alternatives embodiments, the composition of the instant invention does not include an adjuvant.

The invention will now be further illustrated in the following non-limiting example. The example is illustrative only, and is not intended to limit the instant disclosure in any way.

### EXAMPLE 1

### Materials

A batch of ZYLEXIS® produced under commercial conditions was used for this study. ZYLEXIS® consists of the freeze-dried inactivated Parapoxvirus ovis (iPPVO) strain D1701 with an L2 stabiliser and water for injection (WFI). L2 stabilizer contained, per 1 liter, 80 g Dextran 40, 60 g of casein hydrolysate, 80 g of lactose, 130 g of 70% sorbitol solution, and 534 mg of sodium hydroxide.

The freeze-dried pellet (pre-inactivation titer of 7.3 log10 TCID50/ml) was resuspended in 2 mL of water for injection (WFI) just prior to administration. The product was capable of generating a minimum of 1 RP per dose. The control product was Water For Injections (WFI) from the same batch as used to resuspend the product.

The European EHV-1 strain 121412, provided by the Irish Equine Centre (IEC), was used as a challenge strain.

### Methods

Twenty-three Gypsy Cobs arrived on site two weeks before Day 0. Horses were not vaccinated against EHV. Prior to selection and transport to the study site, serum samples were taken to confirm low antibody titers against EHV-1. Information on prestudy screening, routine farm procedures including vaccinations and hygiene procedures, routine medication and diets fed to the horses was kept in the Master Study File (MSF).

The horses were randomly distributed over 2 treatment groups, group T01 (iPPVO-treated; n=11) and group T02 (WFI-treated; n=12). On Days −2, 0 and 7 these animals received an intramuscular injection in the neck with 2 mL ZYLEXIS® or WFI. On Day 0, post treatment administration, horses from both treatment groups were challenged by comingling with EHV-1 infected horses (n=6) which did not receive any treatment and were challenged approximately 3 hours earlier by intranasal aerosol (10^{5.9} TCID₅₀ per animal). On Day 3 horses were diagnosed with a concurrent Strep equi infection.

During the study the general health of all animals was observed and recorded on a daily basis. Both sides of the neck were observed for injection site reactions and rectal temperatures were taken daily from Day −2 through Day 14. Clinical observations related to both EHV-1 challenge and Strep equi infection were made daily from Day 0 through Day 28. These observations included coughing, nasal discharge, ocular discharge, anorexia, depression, dyspnoea and lymph node swelling. Clinical observations specific for Strep equi were made daily from Day 3 through Day 28. These observations included swelling on the lower jaw and swelling of the face.

EDTA blood samples for white blood cell (WBC) counts were collected from Day −2 through Day 21. Nasopharyngeal swab samples for EHV-1 analysis were collected in virus transport medium daily from Day 0 through Day 21. Nasopharyngeal swab samples for Strep equi detection were collected on Days 11, 14, 18, 21, 24 and 28.

Blood samples collected for WBC counts were analyzed at AHVLA Shrewsbury using a Sysmex analyzer. Nasopharyngeal swab samples were tested for the presence of EHV-1 by means of qRT-PCR by the IEC. Nasopharyngeal swab samples for Strep equi were analyzed by Animal Health Trust by means of multiplex PCR.

SAS/STAT User's Version 9.2.2 (SAS Institute, Cary, N.C.) was used for all data analysis. All hypothesis tests were performed at the 0.05 level of significance (two-tailed).

### Results

No injection site reactions or any other adverse events related to the investigational or control product administration were observed during this study.

All clinical observations were considered to be associated with both the EHV-1 challenge and the Strep equi natural infection apart from Lower Jaw Swelling (abscesses) and Facial Swelling which were considered to be characteristic for Strep equi infection.

The incubation period (time between infection and first clinical signs) of Strangles is 7-14 days. It is therefore likely that at least some of the horses were already infected on day −2 when the first dose of inactivated Parapoxvirus was administered, taken into account that the Strep equi typical signs were discovered on day 3 (increased rectal temperature was already present in most of the horses on day 0 prior to the 2^{nd} shot of iPPVO).

Most differences between treatment groups were observed on Day 11 when significantly less nasal discharge, enlarged lymph nodes, EHV-1 virus shedding and lower rectal temperatures were observed in the iPPVO-treated group. In addition, iPPVO -treated horses showed significantly fewer enlarged lymph nodes on Days 17 and 19, significantly less lower jaw swelling on Day 3 and significantly lower rectal temperatures on Days 12 and 13.

In T01 group (iPPVO-treated) a significantly lower percentage of animals as compared to the control group showed abnormal health for Nasal Discharge on Day 11 (P=0.0280); for Enlarged Lymph Nodes on Days 11 (P=0.0352), 17 (P=0.0468) and 19 (P=0.0352) and for Lower Jaw Swelling (Abscesses) on Day 3 (P=0.0352).

On Day 7.2, approximately 4 hours post dosing on Day 7, a significantly lower (P=0.0076) percentage of animals in the control group showed abnormal Nasal Discharge as compared to T01 group while on that same day prior to dosing, no difference was observed between treatment groups.

From Day 3 through Day 22, the percentage of animals showing Lower Jaw Swelling (abscesses) was lower in T01 group as compared to the control group. This difference was significant on Day 3 and was no longer consistently observed as from Day 23 when the Strep equi infection started to resolve. The highest percentage of horses showing abscesses on a given day was 54.5 for T01 group (Days 9 through 19) and 75.0 (Days 7 through 19) for the control group. After controlling for the day of observation, there is very strong evidence (P<0.001) that, on average, animals in T01 group have 2.28 times (95% CI: 1.62, 3.21) the risk of developing abnormal lower jaw swelling compared to the control group. Similar observations were made for Facial Swelling; although no statistical differences were found between treatment groups, from Day 3 through Day 7 and from Day 10 through Day 28 the percentage of animals showing Facial Swelling in T01 group was lower than or equal to the percentage observed in the control group. The highest percentage of horses showing Facial Swelling on a given day was 18.2 for T01 group (Day 9) and 25.0 (Days 6) for the control group. From Day 13 Facial Swelling was no longer observed in the ZYLEXIS® group. On average over all observation days, there is strong evidence (P=0.004) that animals in treatment group 2 have 3.52 times (95% CI: 1.49, 8.29) the odds of developing abnormal facial swelling compared to T01 group.

From Day 0, prior to challenge, horses showed pyrexia. No other signs of abnormal health were recorded and the examining veterinarian recommended that the horses were in sufficient good health to proceed with the challenge. On Day 3 horses were diagnosed with a Strep equi infection for which the first clinical sign of infection is known to be a rapid increase in rectal temperature.

Rectal Temperatures were significantly lower in T01 group as compared to the control group on Days 11 (P=0.0321), 12 (P=0.0440) and 13 (P=0.0180). On these days the percentages of animals with pyrexia were 18.18, 0.00 and 9.09 in T01 group and 50.00, 33.33 and 25.00 in the control group. The back-transformed LSM (±SE) for the percentage of days post-challenge with Pyrexia was 34.03 (±6.13) for the T01 group and 50.15 (±6.31) for the control group. This difference was non-significant (P=0.1551).

From Day 14 through Day 28 the percentage of animals with positive bacterial detection was lower in T01 group than in the control group. On Day 24 this difference was significant (P=0.0477). All animals (direct and in-contact challenged) shed Strep Equi on at least one time-point during the study. The back-transformed LSM (±SE) for the percentage of days shedding was 56.45 (±13.07) in T01 group (range: 16.67 to 83.33) and 86.48 (±11.38) for the controls (range: 16.67 to 100). This difference was not significant (P=0.0844).

Following challenge 7 out of 11 horses in the iPPVO treated group shed low levels of EHV-1 but on Days 11, 12, 13, 14, 15 and 16 quantitative virus detection in this group was significantly lower as compared to the controls. After controlling for the day of sampling, there is very strong evidence (P<0.001) that, on average, animals in T01 group have 2.64 times (95% CI: 1.53, 4.55) the risk of detecting EHV-1 compared to the control group. All animals shed Strep equi but the percentage of animals with positive bacterial detection was lower in T01 group than in the control group from Day 14 through Day 28 with the difference being significant on Day 24. After controlling for the day of sampling, there is some evidence (P=0.021) that, on average, animals in T01 group have 2.50 times (95% CI: 1.15, 5.47) the risk of having strep equi detected compared to the control group.

As it is common that EHV-1 infected horses develop a leukopenia, the drop in WBC counts as compared to the base-line value (measured on Days −2, −1 and 0) was investigated. The duration of a 20% drop in WBC was significantly higher in the iPPVO -treated animals. However, this observation must be seen in the context that the baseline levels were already above the upper limit for a normal range due to the natural Strep equi infection. Therefore the “drop” seen is due to bringing the WBC counts back to normal values. These data are consistent with the function of iPPVO as an immunomodulator which stimulates or counter regulates the immune system according to the needs associated with a specific (stage of) infection.

### Discussion

The clinical observations recorded as from Day 0 can all be attributed to either of the infections: EHV-1 or Strep equi. The first significant difference reported between treatment groups for both infections was for Nasal Discharge on Day 7, 4 hours after administration of the 3^{rd} dose of Parapoxvirus. Because no difference between treatment groups was reported for the occurrence of Nasal Discharge on the same day but prior to test article administration and no differences were reported on this day for any of the other clinical observations, it remains unclear whether this observation is not an artifact. No significant difference was observed on the following day. On Day 11 both Nasal Discharge and Enlarged Lymph Nodes were observed significantly less in the iPPVO-treated group. In contrast to the observation on Day 7, this is considered as a clinical effect of the product because multiple clinical signs including rectal temperature and virus shedding were significantly reduced on Day 11. In addition, iPPVO-treated horses showed significantly less Enlarged Lymph Nodes on Days 17 and 19 as well. It is also worth to mention in this context that, although the difference was not statistically significant due to the low number of observations, three clinical observations, Dypnoea, Depression and Anorexia were never observed in T01 group but were recorded for the control group.

On Days 11, 12 and 13 rectal temperatures were significantly lower for T01 group which indicates a faster recovery as compared to the control group. Clinical Observations typical for Strep equi infection, Lower Jaw Swelling (Abscess) and Facial Swelling were recorded as from Day 3. On this day significantly less iPPVO-treated horses had abscesses. Other observations that illustrate efficacy of iPPVO administration against Strep equi infection are that from Day 3 through Day 22, the percentage of animals showing Lower Jaw Swelling (abscesses) was lower in group of horses treated with iPPVO as compared to the control group and that by Day 13 Facial Swelling was no longer observed in the iPPVO treated group while in the control group two horses still showed Facial Swelling after this time-point.

Following challenge all horses in the direct challenge group, 25% of the horses in the control group and none of the horses in the iPPVO -treated group seroconverted against EHV-1. The lower percentage of sero-conversion for the in-contact challenged group reflects that this type of challenge was milder as compared to the direct intra-nasal challenge. In the iPPVO treated group 7 out of 11 horses did shed low levels of EHV-1 at a certain time-point but none of them did seroconvert while on Days 11, 12, 13, 14, 15 and 16 quantitative virus detection in this group was significantly lower as compared to the controls. This indicates a successful in-contact challenge and efficacy of Parapoxvirus ovis for the reduction of EHV-1 shedding.

It can be speculated that the protective effect of Parapoxvirus ovis is attributed to a stimulation of the innate immune system, which results most probably in an increased stimulation of cell-mediated immunity and in a direct anti-viral effect. This hypothesis is supported by the investigation of Horohov et al. (2008) who showed an increased expression of interferon gamma (IFNγ, indicative for a cell mediated immune response), interferon beta (IFN β, type I IFN with direct antiviral activity), interleukin 15 (IL-15, T-cell growth factor) and interleukin 18 (IL-18, IFNγ inducing factor).

As a consequence of the natural infection with Strep equi, 91% of the iPPVO treated horses and 75% of the control animals respectively had seroconverted for at least one time-point. All animals (direct and in-contact challenged) shed Strep equi on at least one time-point during the study. These data indicate that animals in both pens were exposed to the bacteria. Although on Day 24 significantly fewer horses shed Strep equi in the iPPVO treated group (0%), the percentage of animals with positive bacterial detection was lower in the iPPVO treated group than in the control group from Day 14 through Day 28.

Although the differences for the WBC counts between treatment groups were not significant on any of the investigated days, the duration of marked drop in WBC (leukocytes 80% of pre-challenge values) but not for the duration of leukopenia (leukocytes 60% of prechallenge values) was significantly higher in the iPPVO treated animals. This observation is difficult to interpret because both infections have an opposite effect on the WBC counts (EHV-1 infection decreases while Strep equi infection increases WBC levels) and because the Strep equi infection was already ongoing on Day 0, which caused the WBC levels on Days −2, −1 and 0 to be above the upper limit for a normal range. Therefore the baseline was set too high and the drop seen is due to bringing the WBC counts back to normal values.

In summary, the following can be concluded from this study: administration of inactivated parapoxvirus did not induce injection site reactions, significantly reduced clinical signs related to both EHV-1 and Strep equi infections and significantly reduced EHV-1 and Strep equi shedding.

Every patent and non-patent publication cited in the instant disclosure is incorporated into the disclosure by reference to the same effect as if every publication is individually incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1. A method of protecting a horse in need thereof against a Streprococcus equi equi (Strep equi) infection comprising administering to said horse an immunologically effective amount of Parapoxvirus Ovis.

2. The method of claim 1, wherein said horse is not effectively immunized against Strep equi.

3. The method of claim 1, wherein said horse is infected with EHV-1.

4. A method of protecting a horse against concurrent Strep Equi and EHV infections, comprising administering to said horse an immunologically effective amount of Parapoxvirus ovis.

5. The method of claim 4, wherein said horse is not effectively immunized against Strep equi and EHV.

6. The method of claim 1, wherein said Parapoxvirus ovis is an inactivated Parapoxvirus ovis.

7. The method of claim 1, wherein said Parapoxvirus Ovis comprises Parapoxvirus ovis strain D1701.

8. The method of claim 1, wherein said Parapoxvirus ovis is administered in an aqueous composition.

9. The method of claim 8, wherein said aqueous composition lacks an adjuvant.

10. The method of claim 1, wherein said composition is administered intramuscularly.

11. The method of claim 1, wherein the effective amount of said Parapoxvirus ovis is between 1 RP per dose and 11.6 RP per dose.

12. The method of claim 1, wherein said immunologically effective amount of the Parapoxvirus ovis is administered to the horse three times, and wherein the second administration occurs about 2 days after the first administration, and wherein the third administration occurs about 2 to about 10 days after the second administration.

13. The method of claim 12, wherein the third administration occurs about seven days after the second administration.
